(19) United States
(12) Patent Application Publication
(10) Pub. No.: US 2005/0154429 A1
(43) Pub. Date: Jul. 14, 2005

(54) Acupuncture needle warmer

(76) Inventor: Tien Huang, Arcadia, CA (US)

Correspondence Address: LAW OFFICES OF CLEMENT CHENG, 17220 NEWHOPE STREET #127, FOUNTAIN VALLEY, CA, 92708, US

(21) Appl. No.: 10/754,925

(22) Filed: Jan. 10, 2004

(51) Int. Cl. A61F 7/00; A61F 7/12

(52) U.S. Cl. 607/96; 607/113

(57) ABSTRACT

An acupuncture needle warmer device comprises a flat watertight plastic container filled with supersaturated solution, the container having a closed folded position and an open position, a fold line defined upon the container body wherein the container body can fold on the fold line to snugly enclose a handle of an acupuncture needle in the closed folded position, and an exothermic crystallization trigger located within the container body, triggerable by manual depression.

### BACKGROUND

Traditionally, moxybustion has been used for creating a warm heat source on the coil handle of acupuncture needles. The heat from the top of the needle travels into the bottom of the needle to the patient. Unfortunately, indoor combustion creates pollution and respiratory uneasiness particularly for patients who have allergies.

Non-combustion methods for improving the indoor performance of acupuncture needles have focused on electrical heating and heat lamps. The warm acupuncture needle for treating a patient's body has been modified in many ways. In addition to a wide variety of electrically heated acupuncture needles U.S. Pat. No. 5,250,068 to Ideguchi teaches a light illuminated needle fed from a fiber-optic light source.

### FIELD OF INVENTION

The field of invention is in acupuncture needle moxybustion substitute.

### OBJECT OF THE INVENTION

The object of the invention is to heat an acupuncture needle without combustion or electricity.

### BRIEF DESCRIPTION OF THE DRAWINGS

Figure one is an expanded view of a wide chamber.

Figure two is a half folded view of a wide chamber.

Figure three is a folded view of a thin chamber embodiment.

Figure four is a folded view of the thin chamber embodiment.

Figure five is a cross sectional view of the second embodiment.

Figure six is a cross sectional view of the third embodiment.

### DETAILED DESCRIPTION OF THE PREFERRED EMBODIMENT

An acupuncture needle comprising a phase change heating chamber formed as a cap or bag attaches releaseably to a coil handle of an acupuncture needle. The phase change and heating chamber is liquid and changes to solid state releasing heat transferred to the needle and patient for stimulation of the human nervous system.

Exothermic crystallization from supersaturated solutions provides safe heat. Although a variety of solutions are available, sodium acetate solution is the best mode phase change and heating chamber liquid. NaCH₃COO(aq)=>NaCH₃COO(s). 3H₂O. Sodium acetate can be super cooled and kept in liquid state at room temperature. A user heats the sodium acetate in a container holding the liquid and allows the liquid to cool to a stable state. The liquid can freeze and release heat equal to the phase change heat. The phase change heat increases the temperature of the sodium acetate liquid to a freezing point and subsequently maintains the temperature at that freezing point which is warm and above room temperature. A triggering mechanism such as a metal disk having a plurality of slits cut into the disk has been used for triggering and initiating sodium acetate phase change from liquid to solid.

A semi rigid plastic bag of miniature size preferably contains a small amount of sodium acetate and no air. The sodium acetate is provided in liquid state and can be triggered by a triggering device for phase change to solid state. The plastic bag is preferably flexible and contains at least a main chamber heat sealed with sodium acetate contained in liquid state. Should the liquid state become triggered, the bag housing can be reheated for phase change from solid state to liquid state again. The plastic bag is elongated in shape capable of forming a pocket and enveloping around the coil handle of the acupuncture needle.

For larger amounts of heat, a wide container 10 as shown in figure one has a folding line 30 receiving the coiled handle of the acupuncture needle. A retaining adhesive flap 150 is mounted on the outside surface 50. As shown in figure two the container folds along the folding line 30. The wide container 10 folds on the folding line 30 and is retained in closed position by the adhesive flap that folds over to adhere to the other side of the container. The bag optionally has a thin walled end member 40 allowing greater flexibility.

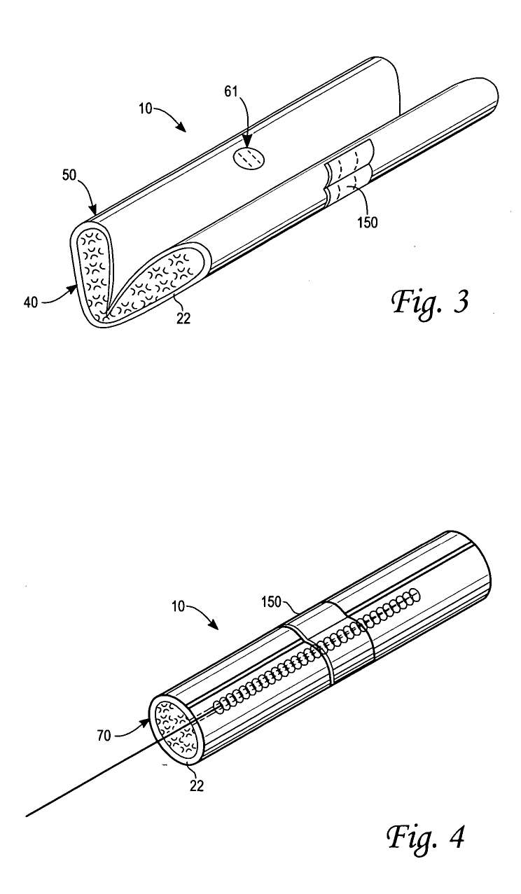

A thin plate triggering device 61 is stored within the container and can be actuated by manual depression on the flexible outside wall. A glass bead, or other ceramic can also be held within the container as a trigger device. For smaller amounts of heat, a less wide thin container can be used as shown in FIG. 3. The thin container also contains a triggering device 61 and adhesive flap 150.

FIG. 4 shows a container in closed position with flap 150 closed around the outside of the container. The end portion 70 maintains a snug fit around the coiled handle.

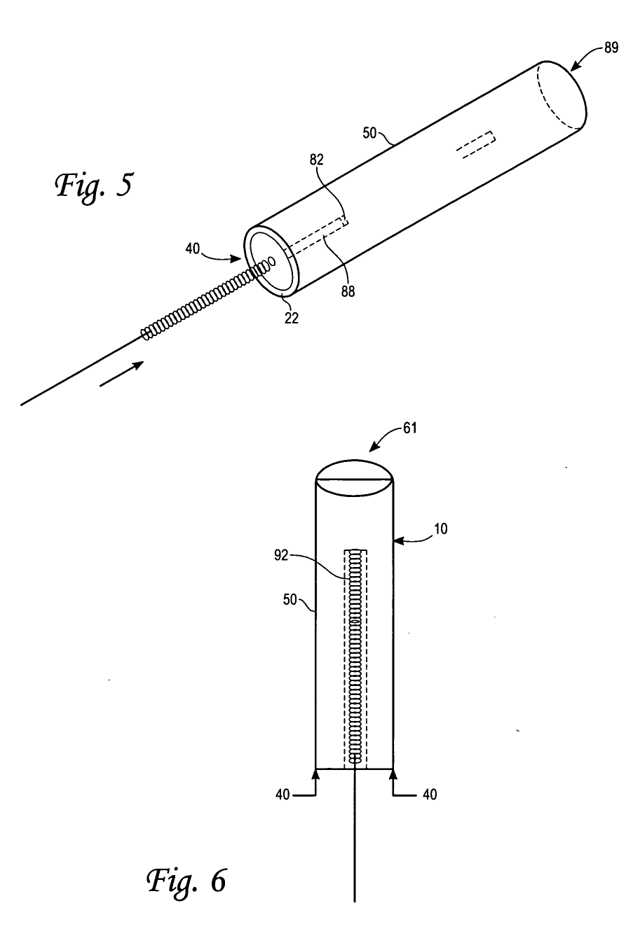

In an alternate embodiment called embodiment No. 2, as shown in FIG. 5, the needle handle punctures a soft membrane 82 that is inside a shallow hollow depression 88 in the container. The depression snugly houses the coiled handle of the acupuncture needle. The needle handle does not exit the opposite end 89. In the needle puncture trigger embodiment, the insertion of the needle handle into the fluid chamber triggers reaction. A soft area 82 allowing penetration into the fluid chamber allows the needle to trigger sodium acetate exothermic crystallization around the needle handle. In this second embodiment, the sidewall 50 should be made of rigid material such as polystyrene.

In an alternate version of embodiment No. 2, the needle can also be implemented with the needle tip puncturing a soft membrane 82 that is inside a hollow depression 88 in the container. The depression snugly houses the coiled handle of the acupuncture needle. The needle tip here exits the opposite end on a second membrane area that allows exit of the needle tip 89. In this needle puncture trigger embodiment, the insertion of the needle into the fluid chamber triggers reaction. A soft area 82 allowing penetration into the fluid chamber allows the needle to trigger sodium acetate exothermic crystallization around the needle. In this second embodiment, the sidewall 50 should also be made of rigid material such as polystyrene. The alternate version of embodiment No. 2 is not preferred because of sterility concerns.

In embodiment No. 3, as shown in FIG. 6, the needle coil handle receives a container formed as a cap 10 fitting snugly upon the coil end of the needle in a cavity 92. A trigger mechanism 61 such as the thin metal plate mechanism is preferably mounted at the tip of the cap. The cavity 92 snugly receives the coiled handle. The end wall 40 has an opening that allows insertion of the coil handle into the cavity 92 before or after activation by trigger mechanism 61. Embodiment No. 3 also has insulation 22 around the cylindrical body has shown in FIG. 6.

In addition to a soft case embodied by a small flexible plastic bag housing holding sodium acetate the invention can also be implemented by using a hard case such as a cylindrical polyethylene housing. The hard housing can be formed as a cap covering the coil handle.

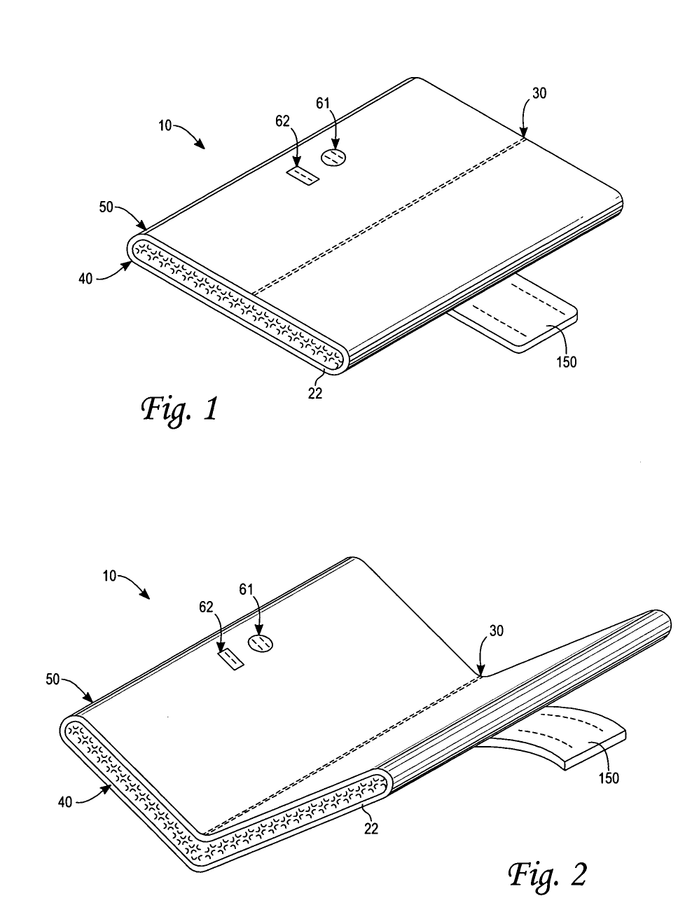

The triggering means is preferably a repeated capable triggering device such as the standard flat planar metal sheet having slits commonly in present use and as described in various patents such as Kapralis U.S. Pat. No. 4,379,448 issued in 1983 and improved versions such as the trigger for a portable heat pack shown in U.S. Pat. No. 6,283,116. As shown on FIGS. 1 and 2, the trigger can also be implemented using standard glass and ceramic activation elements 62 as seen in Milligan U.S. Pat. No. 5,275,156, the reusable heat releasing pack.

Insulation 22 around the exterior of the housing retards heat transfer between the needle, heating chamber and surrounding environment. Insulation could be formed as an air gap between the heating chamber and an outside cover. An outside cover made of expanded polystyrene or other thin soft plastic foam material can be applied to the outside surface.

The insulation can be formed as a sleeve fitting over a cylindrical chamber. The shape of the chamber is preferably cylindrical allowing a sleeve of circular cross section to fit and slide over the chamber. The insulation can be elastic such that it conforms to the outside of the cylindrical chamber. In some embodiments, insulation 22 can be formed as a layer that can be shrunk by heating so that it conforms to the preferably cylindrical and circular cross section.

1. An acupuncture needle warmer device comprising:
a. a flat watertight plastic container filled with supersaturated solution, the container having a closed folded position and an open position,
b. a fold line defined upon the container body wherein the container body can fold on the fold line to snugly enclose a handle of an acupuncture needle in the closed folded position,
c. an exothermic crystallization trigger located within the container body, triggerable by manual depression.

2. The needle warmer device of claim 1 wherein the crystallization trigger is a thin metal plate mechanism having slits that a user can depress through the sidewalls.

3. The needle warmer device of claim 1 further comprising an adhesive tape mounted to the flat watertight plastic container for maintaining the container in closed folded position.

4. The needle warmer device of claim 1 wherein the supersaturated solution is sodium acetate in aqueous form.

5. The needle warmer device of claim 1 wherein the supersaturated solution is sodium acetate in solid form.

6. The needle warmer device of claim 1 wherein the supersaturated solution is held in a cylindrical plastic container.

7. An acupuncture needle warmer device comprising:
a. a rigid watertight plastic container filled with supersaturated solution,
b. a membrane at a first end of the rigid cylindrical watertight plastic container,
c. the membrane can be punctured by an acupuncture needle handle inserted into the rigid watertight plastic container initiating exothermic crystallization of the supersaturated solution around the acupuncture needle handle.

8. The needle warmer device of claim 7 further comprising a crystallization trigger of a thin metal plate mechanism having slits in the rigid watertight plastic container and wherein a shallow depression snugly encloses the handle of the acupuncture needle and holds the membrane mounted inside the hollow depression.

9. The needle warmer device of claim 7 wherein the supersaturated solution is sodium acetate in aqueous form.

10. The needle warmer device of claim 7 wherein the supersaturated solution is sodium acetate in solid form.

11. The needle warmer device of claim 7 wherein the supersaturated solution is held in a cylindrical plastic container.

12. An acupuncture needle warmer device comprising:
a. a rigid watertight plastic container filled with supersaturated solution,
b. a hollow depression at a first end of the rigid cylindrical watertight plastic container, the hollow depression snugly enclosing a handle of an acupuncture needle.
c. an exothermic crystallization trigger located within the container body, triggerable by manual depression.

13. The needle warmer device of claim 12 wherein the crystallization trigger is a thin metal plate mechanism having slits.

14. The needle warmer device of claim 12 wherein the supersaturated solution is sodium acetate in aqueous form.

15. The needle warmer device of claim 12 wherein the supersaturated solution is sodium acetate in solid form.

16. The needle warmer device of claim 12 wherein the supersaturated solution is held in a cylindrical plastic container.
